(19) United States
(12) Patent Application Publication
(10) Pub. No.: US 2015/0094618 A1
(43) Pub. Date: Apr. 2, 2015

(54) AUTOMATED PRESSURE ULCER PREVENTION

(71) Applicant: COVIDIEN LP, Mansfield, MA (US)

(72) Inventors: BRIAN KEITH RUSSELL, Annapolis, MD (US); JONATHAN JAMES WOODWARD, Annapolis, MD (US); DIETRICH OTTO RUEHLMANN, Gaithersburg, MD (US); MARJORIE JONES OLSEN, Charlotte, NC (US); BENJAMIN DAVID MORRIS, Annapolis, MD (US)

(21) Appl. No.: 14/500,156

(22) Filed: Sep. 29, 2014

Related U.S. Application Data
(60) Provisional application No. 61/885,188, filed on Oct. 1, 2013.

(51) Int. Cl. A61B 5/00; A61B 5/11

(52) U.S. Cl. CPC A61B 5/447; A61B 5/1114; A61B 5/746; A61B 5/6801
USPC 600/587

(57) ABSTRACT

Methods, apparatuses and systems are described for monitoring patient position in order to prevent the development of pressure ulcers. The methods may include initiating a first turn timer to calculate a first amount of time spent by a patient in a first position. The methods may further include initiating a first perfusion timer to calculate a second amount of time spent by the patient out of the first position. The methods may then include determining whether to reset the first turn timer based, at least in part, on whether the second amount of time meets or exceeds a predetermined perfusion time threshold. Once the patient has met or exceeded the predetermined perfusion time threshold, an alert may be issued.

### CROSS-REFERENCE TO RELATED APPLICATIONS

This application claims priority to U.S. Provisional Patent Application No. 61/885,188, filed on Oct. 1, 2013, the entirety of which is incorporated by reference herein.

### BACKGROUND

The present disclosure relates generally to a method for preventing pressure ulcers, and in particular, to using a coordinated sensor and timer system to identify when a patient should be moved in order to prevent the formation of pressure ulcers.

Pressure ulcers, more commonly known as bedsores, are preventable injuries that typically occur when the weight of a patient's sedentary body applies prolonged pressure to a localized region of the skin, obstructing blood flow to the soft tissue. The ulcers formed from loss of blood flow may lead to infection and other complications that may be painful and costly to treat, and may ultimately lead to severe injury and even death. Because bedsores and related complications may be classified as preventable hospital-acquired infections, Medicare and some insurance policies may not provide coverage for treatment of bedsores.

Fortunately, bedsores may be easily prevented in many cases simply by turning the patient at regular intervals to relieve the pressure on the soft tissue. By turning the patient, tissue that was previously under pressure is allowed to decompress and blood is permitted to return to the area in a process called perfusion. Traditional methods for preventing bedsores include protocols for nurses to reposition patients several times daily at regular intervals. Such preventative programs may be difficult to monitor, however, and may not be strictly adhered to due to staffing shortages and caregiver oversight. Furthermore, patients who have been properly turned may roll back to their previous position before their compressed tissue has had sufficient time to perfuse, and caregivers may not discover this rollback event until the next scheduled visit, sometimes hours later.

Other known methods for preventing bedsores may utilize sensors positioned at discrete points on a hospital bed mattress, to measure pressure points caused by a patient remaining in one position for a prolonged period of time. These methods, however, only help to avoid bedsores in hospital bed-ridden patients, and do nothing to prevent bedsores in patients in bed rest at home, or who may be spending prolonged periods of time in wheelchairs or other locations besides their hospital beds.

A need may exist, therefore, for a reliable means to automatically monitor patients at-risk for developing bedsores, and to generate an alert when the patient has not received the appropriate preventative care for bedsores.

### SUMMARY

Because bedsores develop quickly in patients spending prolonged periods of time in seated or reclined positions, and due to the difficulties of monitoring each individual at-risk patient to ensure that the patient is turned at regular intervals and does not roll back prematurely, it may be beneficial to provide a reliable means to remotely monitor a patient's position, and to coordinate that position with a set of timers so that alerts may be properly issued when the patient has occupied one position beyond a turn time limit. One method of accomplishing this includes determining, via at least one sensor, that a patient is spending a first amount of time in a first position. It may then be determined, via the at least one sensor, that the patient is spending a second amount of time outside of the first position. The first amount of time may then be reset based on the second amount of time meeting or exceeding a predetermined perfusion time threshold. If the first amount of time meets or exceeds a first predetermined turn time threshold, an alert may be issued.

By monitoring both a first amount of time, correlated to a turn time, and a second amount of time, correlated to a perfusion time, pressure ulcers may be prevented both by avoiding occupying one position beyond a turn time threshold, and ensuring sufficient time for perfusion by requiring that a patient remain out of a first position for a sufficient period of time such that a perfusion time threshold may be met. For example, a patient initially lying on his back may turn to his side in less than the turn time limit. So long as the patient remains on his side for a sufficient amount of time to allow the tissue on his backside to perfuse, he is then free to return to his back, or to remain on his side up until the expiration of the turn time limit for lying on his side.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label with a dash and a second label that distinguishes among the similar components. If only the first reference label is used in the specification, the description is applicable to any one of the similar components having the same first reference label irrespective of the second reference label.

FIG. 1 is a block diagram of an example of a physiological monitoring system in accordance with various embodiments;

FIGS. 2A-2D are graphical representations of methods of preventing pressure ulcers by monitoring one or more patient positions and durations of time spent in each position in accordance with various embodiments;

FIG. 3 is a block diagram of an example of an apparatus in accordance with various embodiments;

FIG. 4 is a block diagram of an example of an apparatus in accordance with various embodiments;

FIG. 5 is a block diagram of an example of a sensing apparatus for receiving physiological data and activity data in accordance with various embodiments;

FIG. 6 is a block diagram of an example of a server for monitoring patient position and duration of time in accordance with various embodiments; and

FIGS. 7 and 8 are flowcharts of various methods for preventing pressure ulcers in accordance with various embodiments.

### DETAILED DESCRIPTION

In order to effectively avoid developing bedsores, both a turn time and a perfusion time may be monitored. In other words, although a patient may be turned from one position to the next at regular intervals, should the patient return to a previous position prior to allowing full perfusion of the tissue, the patient may develop bedsores. Because caregivers cannot realistically monitor patients at all times to ensure that premature rollback does not occur, it is useful to provide a means by which the patient's position may be automatically monitored and correlated to predetermined turn time and perfusion thresholds, such that the development of bedsores may be prevented.

For example, a patient may begin by lying on his back. A caregiver may be scheduled to visit the patient after two hours in order to roll the patient onto his side. However, in some circumstances, the patient may, on his own initiative, roll back to a position on his back at some time after the caregiver has turned the patient to his side. If the patient had only been on his side for ten minutes before he returned to his back, the tissue on his backside may not have had sufficient time to perfuse, such that he is now at risk for developing bedsores. Thus, there may be a benefit to monitoring the patient's position with respect to turn time and perfusion thresholds, such that alerts may be issued if the patient occupies a position for too long, including situations in which a patient returns to a position prematurely without allowing sufficient time for perfusion.

The turn timer and perfusion timer may automatically reset after reaching predetermined thresholds so that patient positions may be monitored on an ongoing basis, regardless of how often the patient turns, or into which position he turns.

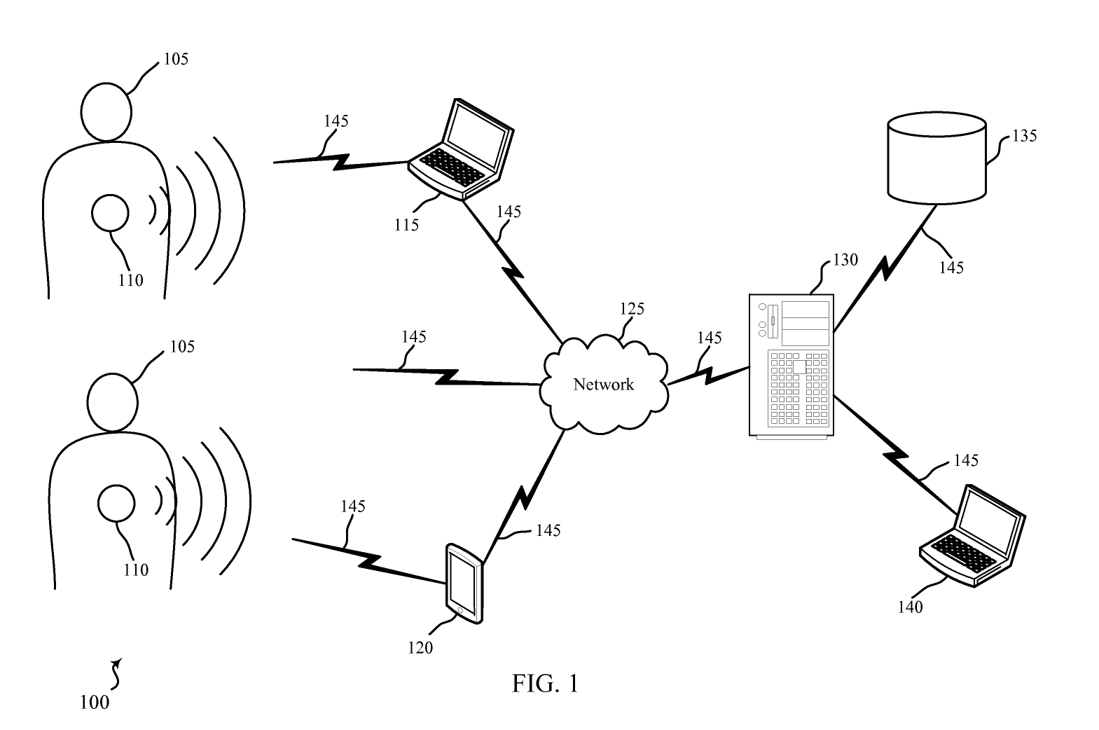

Referring first to FIG. 1, a diagram illustrates an example of a remote physiological measurement monitoring system 100. The system 100 includes patients 105, each wearing a sensor unit 110. The sensor units 110 transmit signals via wireless communication links 145. The transmitted signals may be transmitted to local computing devices 115, 120. Local computing device 115 may be a local caregiver's station, for example. Local computing device 120 may be a mobile device, for example. The local computing devices 115, 120 may be in communication with a server 130 via network 125. The sensor units 110 may also communicate directly with the server 130 via the network 125. The server 130 may be in further communication with a remote computing device 140, thus allowing a caregiver to remotely monitor the patients 105. The server 130 may also be in communication with various medical databases 135 where the collected data may be stored.

The sensor units 110 are described in greater detail below. The sensor units 110 may be a body-worn device, coupled to the patient's chest or to any other suitable portion of the patient's body, such as the patient's arm, thigh, or pelvis. The sensor units 110 may be coupled to the patient using an adhesive, a strap, or any other suitable means. In an alternative embodiment, the sensor units 110 may be coupled to or integral with a garment worn by a patient, such as a belt, wristband, headband, armband, or piece of clothing.

In some embodiments, the sensor units 110 are sensors configured to conduct periodic or ongoing automatic measurements of patient position. A person may wear or otherwise be attached to one or more sensor units 110 so that the sensor units 110 may measure, record, and/or report patient position.

Each sensor unit 110 may be capable of sensing patient position either for the patient's entire body, or for a discrete zone of the patient's body. Multiple sensor units 110 may be used on a single patient. For example, one sensor unit 110 may monitor the position of a patient's hips, while another sensor unit 110 may monitor the position of a patient's shoulders. The data collected by the sensor units 110 may be wirelessly conveyed to either the local computing devices 115, 120 or to the remote computing device 140 (via the network 125 and server 130). Data transmission may occur via, for example, frequencies appropriate for a personal area network (such as Bluetooth or IR communications) or local or wide area network frequencies such as radio frequencies specified by the IEEE 802.15.4 standard.

The sensor units 110 may include any of the sensors, detectors, and/or modules operable to detect a patient's posture, position, and/or orientation as illustrated and described in U.S. Patent Publication No. 2011/0257542, filed Apr. 15, 2011; U.S. Patent Publication No. 2012/0143019, filed Jun. 6, 2011; U.S. Patent Publication No. 2009/0227856, filed Dec. 19, 2008; U.S. Patent Publication No. 2009/0281394, filed Jun. 25, 2009; U.S. Patent Publication No. 2013/0144130, filed Jan. 30, 2012; U.S. Patent Application No. 61/823,596, filed Mar. 15, 2013; U.S. Patent Application No. 61/864,161, filed Aug. 9, 2013; U.S. Patent Application No. 61/823,593, filed Mar. 15, 2013; U.S. Pat. No. 8,400,302, issued Mar. 19, 2013; and/or U.S. Pat. No. 8,079,247, issued Dec. 20, 2011, each of which is commonly owned and which is incorporated herein by reference in its entirety.

In one embodiment, one or more sensor units 110 may comprise an accelerometer to measure patient position data. The accelerometer may be a three-axis microelectromechanical system (MEMS) accelerometer, a piezoelectric accelerometer, a mechanical accelerometer, and/or any other suitable device to detect acceleration and/or static acceleration fields (e.g., the gravitational field). In addition or alternatively, the accelerometer may include a gyroscope operable to detect changes in angular position, angular velocity, and/or angular acceleration of the one or more sensor units 110. The sensor units 110 may be operable to detect the orientation of the sensor unit 110, for example, with respect to a horizontal plane (e.g., the plane defined by the ground surface).

The local computing devices 115, 120 may enable the patient 105 and/or a local caregiver to monitor the collected position measurements. For example, the local computing devices 115, 120 may be operable to present data collected from sensor units 110 in a human-readable format. For example, the received data may be outputted as a display on a computer or a mobile device. The local computing devices 115, 120 may include a processor that may be operable to present data received from the sensor units 110 in a visual format. The local computing devices 115, 120 may also output data in an audible format using, for example, a speaker, or may output data in the form of a haptic alert.

The local computing devices 115, 120 may be custom computing entities configured to interact with the sensor units 110. In some embodiments, the local computing devices 115, 120 and the sensor units 110 may be portions of a single sensor unit 110 operable to sense and display patient position data and data relating to durations of time spent in each position. In another embodiment, the local computing devices 115, 120 may be general purpose computing entities such as a personal computing device, such as a desktop computer, a laptop computer, a netbook, a tablet personal computer (PC), an iPod®, an iPad®, a smart phone (e.g., an iPhone®, an Android® phone, a Blackberry®, a Windows® phone, etc.), a mobile phone, a personal digital assistant (PDA), and/or any other suitable device operable to send and receive signals, store and retrieve data, and/or execute modules.

The local computing devices 115, 120 may include memory, a processor, an output, a data input and a communication module. The processor may be a general purpose processor, a Field Programmable Gate Array (FPGA), an Application Specific Integrated Circuit (ASIC), a Digital Signal Processor (DSP), and/or the like. The processor may be configured to retrieve data from and/or write data to the memory. The memory may be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the local computing devices 115, 120 may include one or more hardware-based modules (e.g., DSP, FPGA, ASIC) and/or software-based modules (e.g., a module of computer code stored at the memory and executed at the processor, a set of processor-readable instructions that may be stored at the memory and executed at the processor) associated with executing an application, such as, for example, receiving and displaying data from sensor units 110.

The data input module of the local computing devices 115, 120 may be used to manually input patient position data and data relating to duration of time spent in each position, instead of or in addition to receiving data from the sensor units 110. For example, a user of the local computing device 115, 120 may make an observation as to a position of the patient and record the observation using the data input module. A user may be, for example, a nurse, a doctor, and/or any other medical healthcare professional authorized to record patient observations, the patient, and/or any other suitable person. For instance, the user may observe that the patient is on his back, and may input this position data into the data input module. In some embodiments, the data input module may be operable to allow the user to select “patient position” and input the observed patient position into the data input module, e.g., using a keyboard. Automatically collected patient position data and data relating to duration of time spent in each position may be used to flag the manually inputted patient position data as having surpassed a predetermined turn time threshold.

The processor of the local computing devices 115, 120 may be operated to control operation of the output of the local computing devices 115, 120. The output may be a television, a liquid crystal display (LCD) monitor, a cathode ray tube (CRT) monitor, speaker, tactile output device, and/or the like. In some embodiments, the output may be an integral component of the local computing devices 115, 120. Similarly stated, the output may be directly coupled to the processor. For example, the output may be the integral display of a tablet and/or smart phone. In some embodiments, an output module may include, for example, a High Definition Multimedia Interface™ (HDMI) connector, a Video Graphics Array (VGA) connector, a Universal Serial Bus™ (USB) connector, a tip, ring, sleeve (TRS) connector, and/or any other suitable connector operable to couple the local computing devices 115, 120 to the output.

As described in additional detail herein, at least one of the sensor units 110 may be operable to transmit patient position data and data relating to duration of time spent in each position to the local computing devices 115, 120 and/or to the remote computing device 140 continuously, at scheduled intervals, when requested, and/or when certain conditions are satisfied (e.g., when the turn timer and/or perfusion timer is operating).

The remote computing device 140 may be a computing entity operable to enable a remote user to monitor the output of the sensor units 110. The remote computing device 140 may be functionally and/or structurally similar to the local computing devices 115, 120 and may be operable to receive data streams from and/or send signals to at least one of the sensor units 110 via the network 125. The network 125 may be the Internet, an intranet, a personal area network, a local area network (LAN), a wide area network (WAN), a virtual network, a telecommunications network implemented as a wired network and/or wireless network, etc. The remote computing device 140 may receive and/or send signals over the network 125 via communication links 145 and server 130.

The remote computing device 140 may be used by, for example, a health care professional to monitor the output of the sensor units 110. In some embodiments, the remote computing device 140 may receive an indication of one or more patient position data and data relating to duration of time spent in each position when the sensor units 110 detect that the turn timer and/or perfusion timer is running, when the healthcare provider requests the information, at scheduled intervals, and/or at the request of the patient 105. For example, the remote computing device 140 may be operable to receive patient position data and data relating to duration of time spent in each position from the server 130 and display the position and duration of time data in a convenient format. The remote computing device 140 may be located, for example, at a nurses station or in a patient's room, and may be configured to display position and duration of time data collected from one or more patients 105. In some instances, the local computing devices 115, 120 may also be operable to receive and display patient position and duration of time data in much the same way that the remote computing device 140 is operable. In some embodiments, the remote computing device 140 may be operable to receive an indication, based on the output of one or more sensor units 110, that an alert should be issued. The alert issued at the remote computing device 140 may be any one or more of a visual, auditory, or haptic alert.

The server 130 may be configured to communicate with the sensor units 110, the local computing devices 115, 120, the remote computing device 140 and databases 135. The server 130 may perform additional processing on signals received from the sensor units 110 or local computing devices 115, 120, or may simply forward the received information to the remote computing device 140 and databases 135. The databases 135 may be examples of electronic health records (“EHRs”) and/or personal health records (“PHRs”), and may be provided by various service providers.

Additionally, although the remote computing device 140 and the local computing devices 115, 120 are shown and described as separate computing devices, in some embodiments, the remote computing device 140 performs the functions of the local computing devices 115, 120 such that a separate local computing device 115, 120 may not be necessary. In such an embodiment, the user (e.g., a nurse or a doctor) may manually enter the patient's position data and data relating to duration of time spent in each position directly into the remote computing device 140.

In the system 100 of FIG. 1, a sensor unit 110 may detect patient position data and data relating to duration of time spent in each position. In some embodiments, a single sensor unit 110 may detect both patient position data and data relating to duration of time spent in each position. In alternate embodiments, one sensor unit 110 may detect patient position data, while a second sensor unit 110 may detect duration of time data. In addition, one or more sensor units 110 may detect patient position data and timer data for discrete zones of the patient's body. For example, a first sensor unit 110 may detect patient position and timer data for a patient's shoulders, while a second sensor unit 110 may detect patient position and timer data for a patient's hips. In this way, if a patient turns one portion of his body without relieving pressure from another portion of his body, separate turn timers and perfusion timers may be maintained to ensure proper perfusion of each portion.

Based on the received patient position data and data relating to duration of time spent in each position, it may be determined whether the patient has occupied one position beyond a predetermined turn time threshold, or whether the patient has returned to a position before reaching a predetermined perfusion time threshold. This determination may be made at the one or more sensor units 110, or may be determined at any one of the local computing devices 115, 120, the remote computing device 140, or the server 130. Patient position data and data relating to duration of time spent in each position may be received on an ongoing basis from the one or more sensor units 110. Upon determining that the patient has occupied one position for a length of time meeting or exceeding the predetermined turn time threshold, including if the patient has returned to a position before the predetermined perfusion time threshold has been met or exceeded, an alert may be issued. This alert may be transmitted to the patient or clinician to be displayed, for example, at a nurses station or in a patient's room, or alternatively on any of the local computing devices 115, 120 or the remote computing device 140. The alert may notify the patient and/or caregiver that the patient is at risk of developing bedsores, and should be turned to a new position.

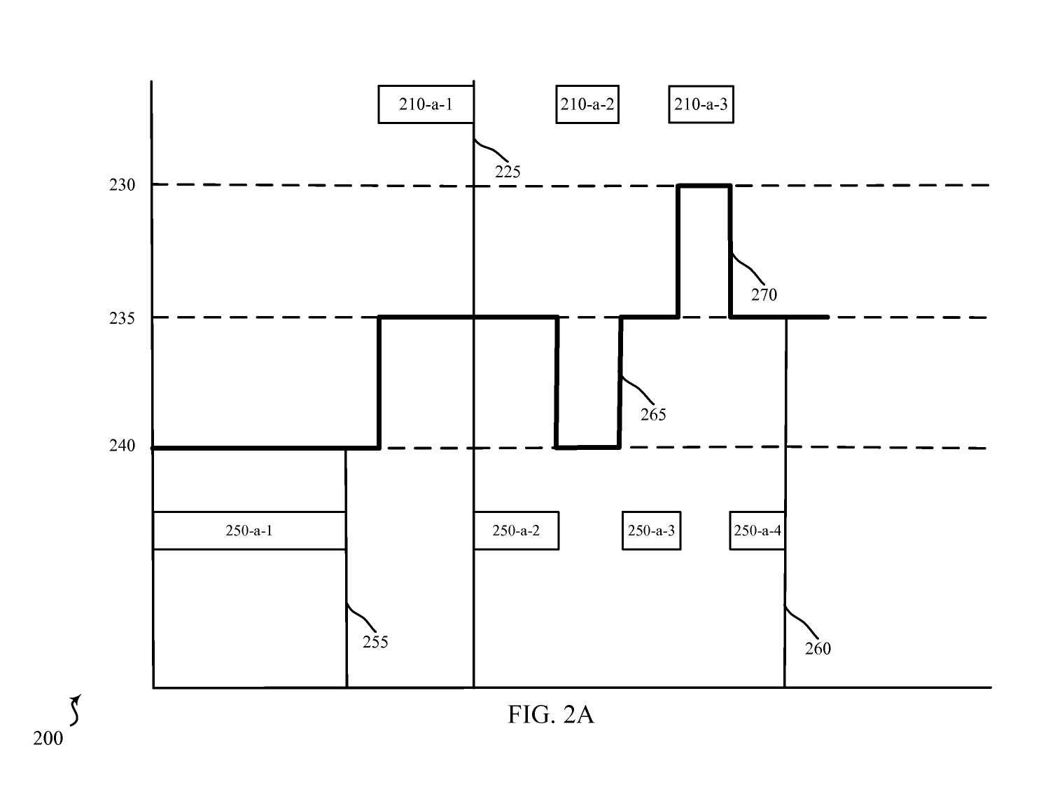

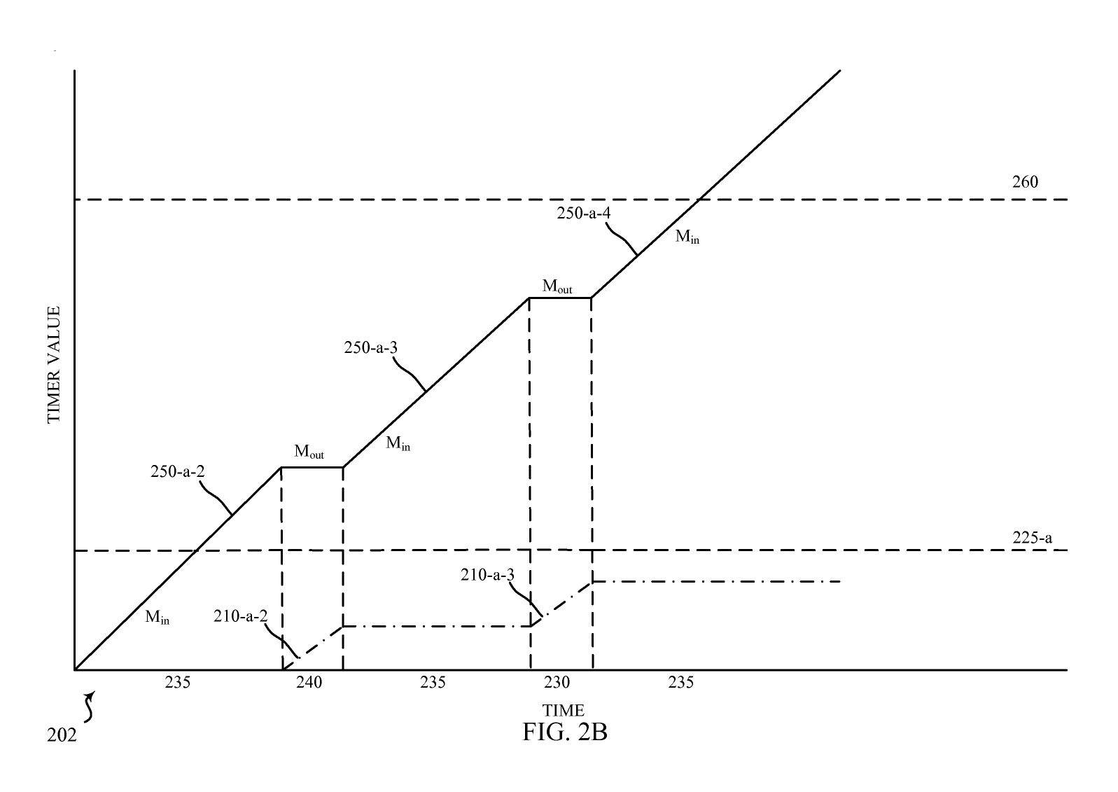

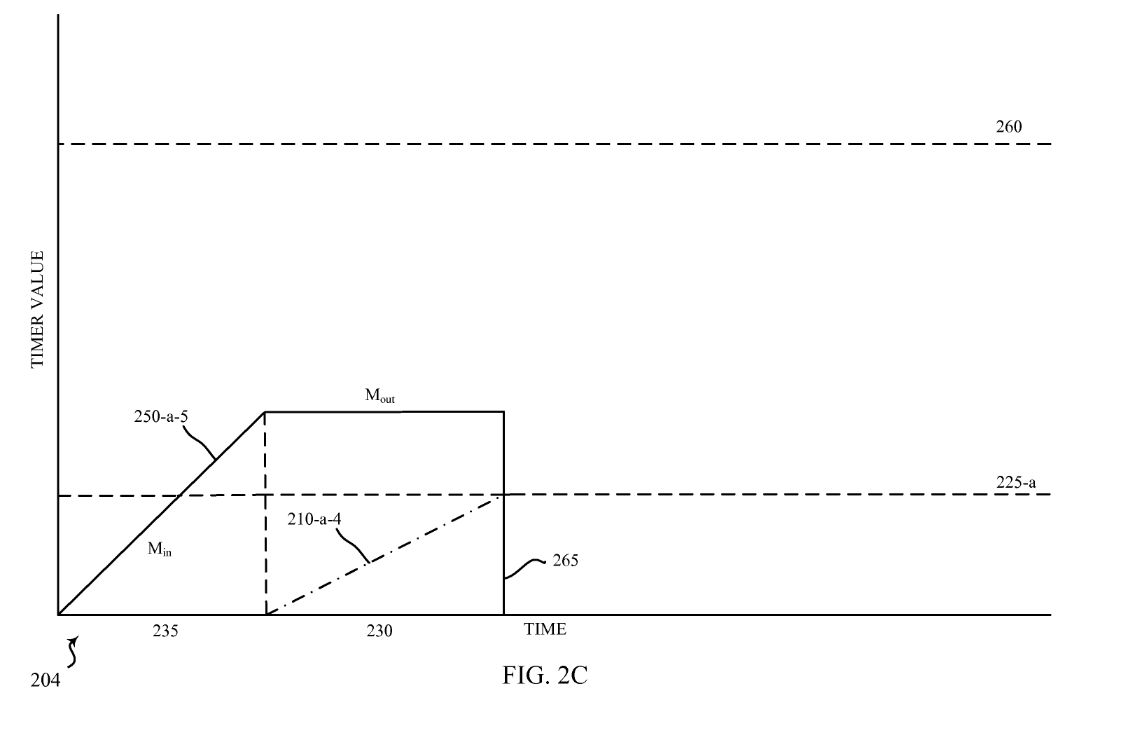

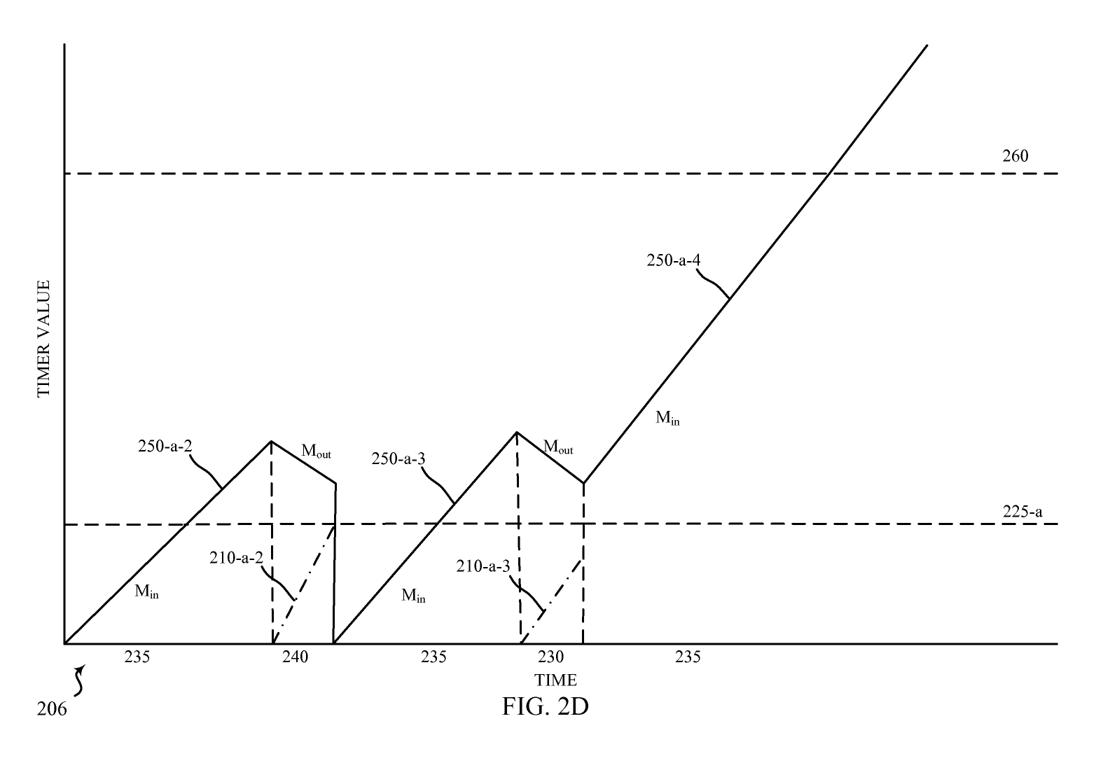

FIG. 2A is a graphical representation 200 of an example process by which patient position may be monitored with respect to predetermined turn time and perfusion time thresholds, wherein patient position is shown on the vertical axis and time is shown on the horizontal axis. Patient position data and data relating to duration of time spent in each position may be collected by one or more sensor units 110 as shown in FIG. 1. As shown in the example of FIG. 2A, the patient may begin in a first position 240, for example on his back. The patient remains in the first position 240 as measured by a turn timer operation 250-a-1, for a duration of, for example, two hours, and therefore an alert is issued once the turn time limit 255 has been reached. A caregiver receives the alert, or the patient is notified of the alert, and the patient is rolled into a second position 235, for example onto his right side. The patient remains in the second position 235 for a duration of time as measured by a perfusion timer operation 210-a-1, which may be, for example, twenty minutes, such that his back tissue has had sufficient time to perfuse upon reaching the perfusion time limit 225. After the perfusion time limit 225 has been reached, the perfusion timer and the turn timer reset. The turn timer operation 250-a-2 then begins while the patient remains in the second position 235. Before the turn timer operation 250-a-2 has reached the turn time limit 260, the patient returns from the second position (his right side) 235 to the first position (his back) 240. Upon leaving the second position 235, the turn timer operation 250-a-2 pauses, and the perfusion timer operation 210-a-2 begins. Before the perfusion timer operation 210-a-2 has reached the perfusion time limit 225, however, the patient experiences a rollback event 265, in which he returns to the second position 235, and the turn timer operation 250-a-3 starts up again where turn timer operation 250-a-2 left off. Before the turn timer operation 250-a-3 has reached the turn time limit 260, the patient then rolls to a third position 230, for example onto his left side. Upon entering the third position 230, the turn timer operation 250-a-3 pauses, and perfusion timer operation 210-a-3 begins. Before the perfusion timer operation 210-a-3 has reached the perfusion time limit 225, however, the patient experiences a second rollback event 270, in which he returns to the second position 235, and the turn timer operation 250-a-4 again begins to run, picking up where turn timer operation 250-a-3 left off. The patient remains in the second position 235 beyond the turn time limit 260, because turn timer operations 250-a-2, 250-a-3, 250-a-4 in aggregate calculates the total time spent by the patient in the second position 235. Accordingly, an alert is issued to indicate to the patient and/or caregiver that the patient has occupied the second position 235 for too long and should again be rolled. In this way, the position sensor and timers system is able to monitor total time spent in a single position, regardless of any number of rollback events.

Although illustrating only three positions, the methods described herein may be used to calculate turn time thresholds and perfusion time thresholds for any number of patient positions. In addition, a turn timer and perfusion timer set may run independently for discreet zones on a patient's body, as described above. For example, a first turn timer and perfusion timer may begin to operate for a patient's tailbone, and a second turn timer and perfusion timer may operate for a patient's shoulder.

In addition, turn timers and/or perfusion timers may operate at various rates M with respect to each other and/or to various zones on a patient's body, as illustrated in FIGS. 2B-2D. The graphical representation 202 of FIG. 2B, for example, illustrates the operation of turn timer 250 and perfusion timer 210 during a time period that corresponds to that period of time in FIG. 2A between a time when the turn time limit 255 is met and a time when the turn time limit 260 is met. Thus, reference numbers in FIG. 2B may correspond to reference numbers in FIG. 2A. For example, as shown in FIG. 2B, a turn timer operation 250-a-2 may advance at a rate of, for example, Mᵢₙ, while the patient is in a second position 235. In an example, Mᵢₙ, the rate at which the turn timer operates when the patient is in a given position, may be set equal to 1, 2, etc. When the patient rolls out of second position 235 into a first position 240, the turn timer may operate at a rate of Mₒᵤₜ, the rate at which the turn timer operates when the patient is out of the given position. In the example of FIG. 2B, the rate Mₒᵤₜ may be set to zero, or in other words, the turn timer may pause for so long as the patient is out of the second position 235. When the patient returns to the second position 235, the turn timer operation 250-a-3 may again advance at a rate of Mᵢₙ, for so long as the patient remains in the second position 235. Again, when the patient rolls out of the second position 235 into a third position 230 (which may or may not be the same position as first position 240), the turn timer operation 250-a-3 pauses, or advances at a rate of Mₒᵤₜ. And again, when the patient rolls back into the second position 235, the turn timer operation 250-a-4 again advances at a rate of Mᵢₙ until the turn time limit 260 is reached, at which point an alert is issued. Meanwhile, a perfusion timer operation 210-a-2 may be used to measure periods of time spent out of the second position 235. For example, when the patient rolls out of the second position 235 into a first position 240, a perfusion timer operation 210-a-2 begins. However, when the patient returns to the second position 235 before the perfusion timer operation 210-a-2 has reached the perfusion time limit 225-a, the perfusion timer operation 210-a-2 pauses, or advances at a rate of zero. In alternate embodiments, the perfusion timer operation 210-a-2 may advance at a negative rate for the duration of time spent by the patient in the second position (see, for example, FIG. 2D, described below). Similarly, when the patient moves out of the second position 235 into a third position 230 (which may or may not be the same position as first position 240), the perfusion timer operation 210-a-3 again begins. But again, when the patient rolls back into the second position 235 before the perfusion timer operation 210-a-3 has reached the perfusion time limit 225-a, the perfusion timer operation 210-a-3 pauses. In this way, regardless of how many times a patient switches positions in any given period of time, the patient may be monitored remotely and automatically to avoid the development of bedsores without the need for constant caregiver presence and attention.

In an alternate embodiment not illustrated in FIG. 2B, the perfusion timer operations 210-a-2, 210-a-3 may, rather than pausing or operating at a rate of zero when the patient moves back to the second position 235, instead reset such that the perfusion timer operations 210-a-2, 210-a-3 begin from an initialized state when the patient returns to the first position 240 or third position 230. In this way, a patient may only be determined to have had sufficient time to perfuse when he remains out of a position for a continuous period of time meeting or exceeding a perfusion time limit, as opposed to being given credit for a plurality of discrete periods of time spent out of a position that in the aggregate meet or exceed the perfusion time limit. The determination of whether to allow for perfusion in the aggregate or to require continuous perfusion may be made with respect to each patient's individual physiological parameters.

Although illustrated as advancing at a linear rate, either or both the perfusion timer and turn timer may advance linearly, non-linearly, or via a step function. The rates and thresholds of each or both of the perfusion timer and turn timer may be predetermined by a patient or caregiver in conjunction with the patient's individual physiological parameters, or by any means necessary to ensure individually-tailored parameters for avoiding the development of pressure ulcers. For example, age and dehydration may affect the rate at which bedsores develop. Additionally, the perfusion timer and turn timer may operate at different rates.

In one embodiment illustrated in the graphical representation 204 of FIG. 2C, both the perfusion timer and the turn timer may reset when the perfusion timer reaches a predetermined perfusion time limit. For example, the patient may be sitting or lying down in a second position 235, during which time a turn timer operation 250-a-5 may advance at, for example, a rate of Mᵢₙ, which in some examples may be equal to 2. When the patient shifts into a third position 230, the perfusion timer operation 210-a-4 may begin. When the perfusion timer operation 210-a-4 reaches the perfusion time limit 225-a, although the turn timer operation 250-a-5 has not yet reached the turn time limit 260, both the perfusion timer and the turn timer may reset. This is because the patient has occupied the third position 230 for a sufficient amount of time to allow the tissue previously under pressure in second position 235 to perfuse, such that the system may now monitor the length of time spent by the patient in the third position 230 before the turn time limit 260 is reached.

In an embodiment, each of the perfusion time limit and turn time limit may be predetermined and/or adjusted on the basis of individual patient physiological data or industry standards. The turn time limit may also be determined to allow for overlap between the perfusion time and the turn time. For example, as shown in FIG. 2C, when the patient moves into the third position 230, the turn timer operation 250-a-5 will be paused, or in other words operate at a rate of Mₒᵤₜ=0, until the perfusion time limit 225-a is reached, at which point the perfusion timer and the turn timer may both reset. After the turn timer has reset, the turn timer operation 250-a-5 or a second turn timer operation (not shown) will begin to measure the length of time spent by the patient in the third position 230; however, the patient will have already spent the duration of the perfusion timer operation 210-a-4, for example twenty minutes, in the third position 230 before the turn timer operation 250-a-5 has restarted or the second turn timer (not shown) has started to operate. Thus, the turn time limit 260 may be predetermined to account for this twenty minute lag in time, such that the patient does not spend more than a predetermined time, after which he risks developing bedsores, in any given position.

In an alternative embodiment, the turn timer may operate at a positive rate when the patient is in a second position, and may operate at a negative rate when the patient is out of the second position, as illustrated in the graphical representation 206 of FIG. 2D. For example, a patient may be in a second position 235, during which time the turn timer 250-a-2 is operating at a positive rate of, for example, Mᵢₙ. When the patient shifts out of the second position 235 into a first position 240, the turn timer operation 250-a-2 may advance at a negative rate of Mₒᵤₜ for the duration of time the patient spends out of the second position 235. In some embodiments, Mᵢₙ may be equal to a positive rate of 1, while Mₒᵤₜ may be equal to a negative rate of −1. In other embodiments the rates may be alternate values. Additionally, when the patient turns out of the second position 235 into the first position 240, a perfusion timer operation 210-a-2 may begin to measure the time spent by the patient out of the second position 235. In the example illustrated in FIG. 2D, the patient remains in the first position 240 for a sufficient period of time to allow the tissue to perfuse, and the perfusion time limit 225-a is reached. When the perfusion time limit 225-a is reached, both the turn timer and the perfusion timer may reset. In this example, the patient returns to the second position 235 upon reaching the perfusion time limit 225-a, such that the turn timer operation 250-a-3 is again advancing at a positive rate of Mᵢₙ. Before the turn timer operation 250-a-3 has reached the turn time limit 260, the patient again shifts out of the second position 235 into a third position 230, which may or may not be the same position as first position 240. While the patient is in the third position 230, the turn timer operation 250-a-3 may advance at a negative rate of Mₒᵤₜ for the duration of time the patient spends out of the second position 235. At the same time, when the patient turns out of the second position 235 into the third position 230, a perfusion timer operation 210-a-3 may begin to measure the time spent by the patient out of the second position 235. In this example, the patient shifts back into the second position 235 prior to the perfusion timer operation 210-a-3 reaching the perfusion time limit 225-a, such that the turn timer will not reset. Thus, when the patient shifts back into the second position 235, the turn timer operation 250-a-4 will continue to advance at a positive rate of Mᵢₙ to measure the period of time spent by the patient in the second position 235. In the illustrated example, the time spent by the patient in the second position 235 exceeds the predetermined turn time limit 260, and an alert is issued.

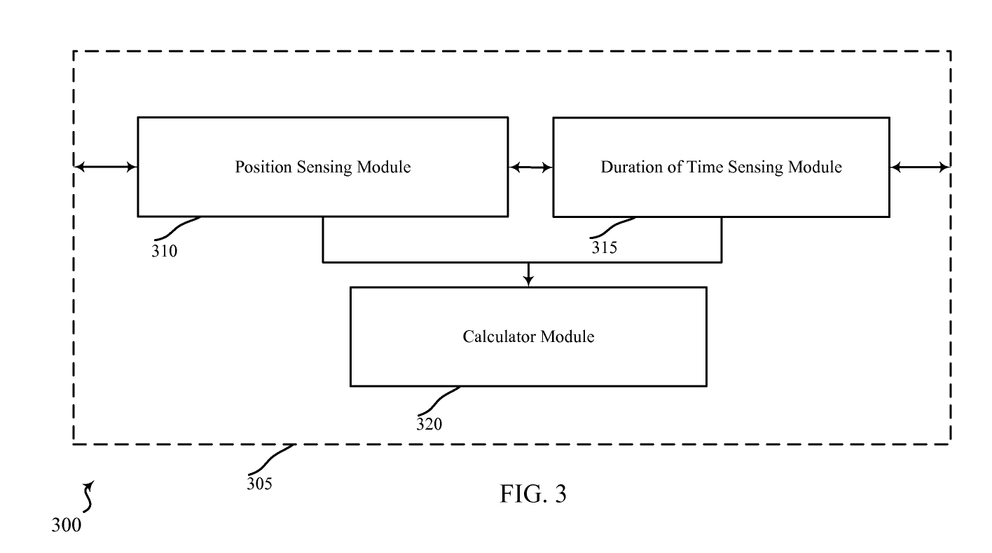

FIG. 3 shows a block diagram 300 that includes apparatus 305, which may be an example of one or more aspects of the local computing devices 115, 120 and/or remote computing device 140 (as shown in FIG. 1) for use in monitoring a patient's one or more positions and duration of time spent in each position, in accordance with various aspects of the present disclosure. In an alternative embodiment, apparatus 305 may be an aspect of one or more sensor units 110. In some examples, the apparatus 305 may include a position sensing module 310, a duration of time sensing module 315, and a calculator module 320. Each of these components may be in communication with each other.

The components of the apparatus 305 may, individually or collectively, be implemented using one or more application-specific integrated circuits (ASICs) adapted to perform some or all of the applicable functions in hardware. Alternatively, the functions may be performed by one or more other processing units (or cores), on one or more integrated circuits. In other examples, other types of integrated circuits may be used (e.g., Structured/Platform ASICs, Field Programmable Gate Arrays (FPGAs), and other Semi-Custom ICs), which may be programmed in any manner known in the art. The functions of each unit may also be implemented, in whole or in part, with instructions embodied in a memory, formatted to be executed by one or more general or application-specific processors.

The position sensing module 310 may be configured to monitor the position of a patient, as the patient remains in or shifts to one or more positions, where the determined positions may be based, at least in part, on the position data received from one or more sensor units 110. The duration of time sensing module 315 may be configured to monitor the duration of time spent by the patient in each position. The duration of time sensing module 315 may sense one or more periods of time concurrently and/or sequentially. For example, the duration of time sensing module 315 may monitor the duration of time spent by the patient in a first position, and may pause the timer for the first position and monitor the duration of time spent by the patient a second position, and may further continue to operate the timer for the first position when the patient shifts back into the first position, etc.

Calculator module 320 may be configured to determine whether any of a predetermined turn time threshold or predetermined perfusion time threshold has been met or surpassed based on data received from the position sensing module 310 and duration of time sensing module 315. Based on this determination, calculator module 320 may provide an alert or other visual, auditory or haptic notification either to the patient locally at apparatus 305, or may transmit an alert to either the local computing devices 115, 120 or the remote computing device 140 via the network 125 and server 130, where the alert may be received by a caregiver. Additionally, the calculator module 320 may determine that one or more of the turn timer and perfusion timer should be reset based, at least in part, on one or more of the turn time threshold or perfusion threshold being met or surpassed.

In some examples, apparatus 305 may be operable to receive data streams from the sensor units 110, as well as to send and/or receive other signals between the sensor units 110 and either the local computing devices 115, 120 or the remote computing device 140 via the network 125 and server 130. In one embodiment, apparatus 305 may receive data streams from the sensor units 110 and also forward the data streams to other devices. Apparatus 305 may include wired and/or wireless connectors. For example, in some embodiments, sensor units 110 may be portions of a wired or wireless sensor network, and may communicate with the local computing devices 115, 120 and/or remote computing device 140 using either a wired or wireless network. Apparatus 305 may be a wireless network interface controller (“NIC”), Bluetooth® controller, IR communication controller, ZigBee® controller and/or the like. In alternate embodiments, apparatus 305 may be a component of one or more sensor units 110 such that patient position and duration of time data received at one or more sensor units 110 may be processed by apparatus 305 at the one or more sensor units 110, and may be either displayed or translated into an alert at apparatus 305, or alternatively may be transmitted to either the local computing devices 115, 120 or the remote computing device 140 via the network 125 and server 130, which may in turn issue an alert.

The local computing device 115, 120 and/or remote computing device 140, upon receiving a signal from apparatus 305, may send alerts using such methods as short message service (SMS) text messages, email, or any other suitable means. For example, if the signal indicates that a patient has occupied a position beyond a predetermined turn time threshold, the monitoring station may send information to the patient, a clinician, support personnel, a family member, etc. The information may alert the patient or caregiver that the patient is at risk for developing pressure ulcers, and should be shifted to a different position.

In some embodiments in which apparatus 305 is a component of one or more sensor units 110, apparatus 305 may be operable to determine when a local computing device 115, 120 and/or remote computing device 140 is available to receive a signal from apparatus 305. For example, apparatus 305 may detect when a local computing device 115, 120 and/or remote computing device 140 is within a certain distance of the apparatus 305. In such an embodiment, the transceiver module 325 may push data to the local computing device 115, 120 and/or remote computing device 140. In other embodiments, data may be pulled from apparatus 305 by the local computing device 115, 120 and/or remote computing device 140. In other words, apparatus 305 may receive a signal requesting patient position and duration of time data from the local computing device 115, 120 and/or remote computing device 140.

In some examples, apparatus 305 may include circuitry, logic, hardware and/or software for processing the data streams received from the sensor units 110. Apparatus 305 may include filters, analog-to-digital converters and other digital signal processing units. Data processed by a signal processing module may be stored in a buffer, for example, in a storage module. The storage module may include magnetic, optical or solid-state memory options for storing data processed by the signal processing module. The position sensing module 310 may access the data stored in the storage module and output a signal associated with the patient position data.

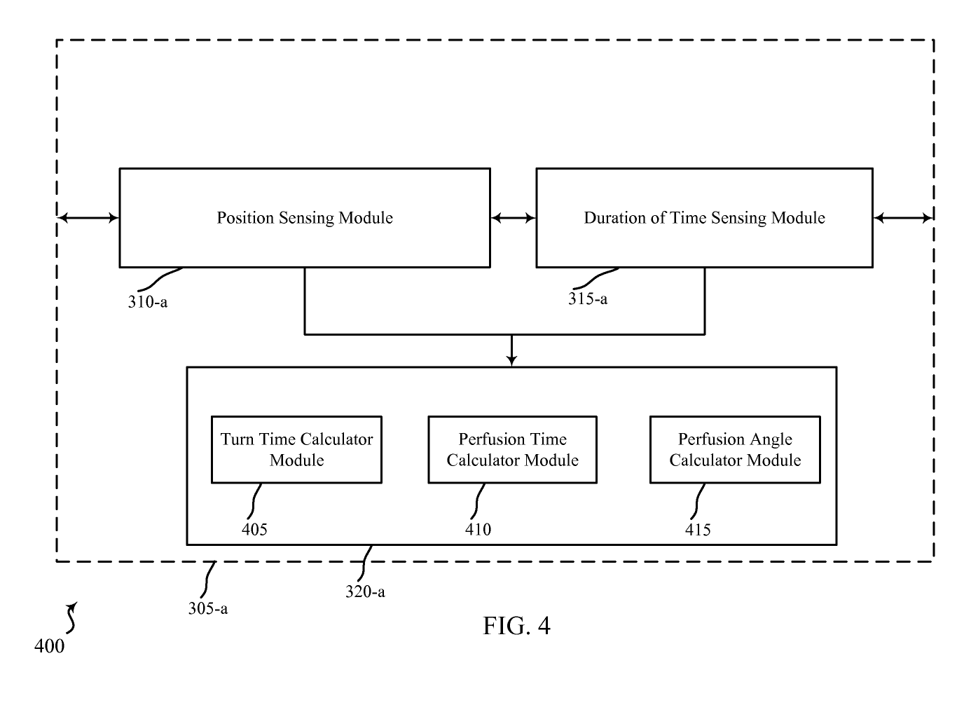

FIG. 4 shows a block diagram 400 that includes apparatus 305-a, which may be an example of apparatus 305 (as illustrated in FIG. 3), in accordance with various aspects of the present disclosure. In some examples, apparatus 305-a may include a position sensing module 310-a, a duration of time sensing module 315-a, and a calculator module 320-a, which may be examples of the position sensing module 310, duration of time sensing module 315, and calculator module 320 of FIG. 3, respectively. In some examples, the calculator module 320-a may include a turn time calculator module 405, a perfusion time calculator module 410, and a perfusion angle calculator module 415. The modules 405, 410 and/or 415 may each be used in aspects of correlating monitored patient position data and duration of time data with predetermined turn time, perfusion time, and perfusion angle thresholds. Additionally, while FIG. 4 illustrates a specific example, the functions performed by each of the modules 405, 410 and/or 415 may be combined or implemented in one or more other modules.

The turn time calculator module 405 may be used to correlate patient position data received from the position sensing module 310-a with duration of time data received from the duration of time sensing module 315-a in order to calculate the amount of time spent by the patient in one or more positions. As discussed above with respect to FIGS. 2A-D, a duration of time spent in a given position may consist of a single, ongoing period of time spent in the position, or may comprise a summation of a plurality of distinct time periods spent in a position as the patient shifts between various positions. For example, a patient may remain on his back continuously for two hours, or may alternatively remain on his back for 30 minutes, shift to his side for 5 minutes, return to his back for 40 minutes, shift to his other side for 10 minutes, return to his back for 5 minutes, shift to his side for 15 minutes, return to his back for 40 minutes, shift to his side for 10 minutes, and return to his back for 5 minutes, such that in the aggregate, the patient has spent a total of two hours on his back. If, for example, each shift out of the first position failed to reach a predetermined perfusion time threshold, for example twenty minutes, the turn timer would continue to run such that the patient has reached a turn time limit of two hours in the aggregate. In this way, the duration of time spent in a given position may be a summation of time calculated from a plurality of individual time periods. The rate of the turn timer operated by the turn time calculator module 405 may operate at a positive rate as illustrated in FIG. 2B, or at a negative rate, as illustrated in FIG. 2C, or may operate at a zero rate, which may correspond to a paused turn timer, as also illustrated in FIG. 2B. The turn time calculator module 405 may then correlate the duration of time spent in a given position to a predetermined turn time threshold to determine whether the patient has occupied the given position beyond the turn time threshold. If the patient occupies the given position up to or beyond the predetermined turn time threshold, the turn time calculator module 405 may issue an alert directly at apparatus 305-a, or may transmit a signal to a local computing device 115, 120 or remote computing device 140 to provide an alert to the patient or to a caregiver, indicating that the patient should be turned to a new position.

The perfusion time calculator module 410 may similarly be used to correlate patient position data received from the position sensing module 310-a with duration of time data received from the duration of time sensing module 315-a in order to calculate the amount of time spent by the patient out of one or more positions. As discussed above with respect to FIGS. 2A-D, the perfusion timer may operate only for a single continuous period of time spent by the patient out of a given position, or may operate as a summation of time spent by a patient out of a given position over a plurality of periods of time. When the patient returns to the given position, the perfusion timer will pause, or operate at a zero rate, until the patient again shifts out of the given position. For example, as illustrated in FIG. 2A, the perfusion timer 210-a-2 may begin to operate when the patient leaves a second position 235, but may discontinue operating, or pause, when the patient returns to the second position 235. The perfusion timer 210-a-3 may then continue to operate when the patient again leaves the second position 235, but may again pause when the patient shifts back to the second position 235. In this way, the perfusion timer may reach or exceed a predetermined perfusion time threshold when the patient remains out of a given position for a plurality of discrete periods of time. In other embodiments, the patient may remain out of a given position for one continuous period of time, such that the continuous period of time reaches or exceeds a predetermined perfusion time limit. The perfusion time calculator module 410 may thus use the data collected from position sensing module 310-a and duration of time sensing module 315-a to determine that a patient is out of a given position, and to correlate the amount of time spent by the patient out of the given position to a predetermined perfusion time threshold to determine whether the patient has properly achieved perfusion for the given position. Upon reaching or exceeding the predetermined perfusion time threshold, the perfusion timer may reset and may begin to operate again when the patient again shifts out of a given position.

The perfusion angle calculator module 415 may use data provided by the position sensing module 310-a to determine whether a patient has shifted out of a given position. For example, while a patient may roll from his back to his side, if he does not roll fully onto his side such that all of the tissue previously placed under pressure while he was on his back is given the opportunity to perfuse, the patient cannot be considered to have left his back for purposes of determining a risk of developing bedsores. For example, if there is only a small difference, for example a close to 0° difference, a 5° difference, or a 10° difference, between the first and second positions, the patient may not have changed position enough to allow the tissue that was compressed in the first position to perfuse. Rather, it may be necessary for the patient to turn at least 15°, at least 20°, or at least 45°, for example, to allow the tissue compressed in the first position to perfuse. Thus, the perfusion angle calculator module 415 may correlate patient position data received from the position sensing module 310-a with a predetermined perfusion angle threshold to determine whether the patient has in fact shifted out of a first position into a second position. The perfusion angle calculator module 415, in conjunction with the position sensing module 310-a, may be operable to detect the angle at which the patient is seated or lying down based on the detected direction of the gravitational field. If the perfusion angle calculator module 415 determines that the patient position data has reached or exceeded the predetermined perfusion angle threshold, the turn timer for the first position may, in some embodiments, pause or operate at a zero rate, or in other embodiments, may operate at a negative rate, for the duration of time spent by the patient out of the first position. In addition, upon determining that the patient has left a first position, the perfusion timer may operate for the duration of time spent by the patient out of the first position. In the alternative, if the perfusion angle calculator module 415 determines from the data received from the position sensing module 310-a that the patient has not shifted sufficiently to meet the predetermined perfusion angle threshold, the turn timer for the first position will continue to operate at a positive rate until such time as the patient is determined to have shifted out of the first position by the position sensing module 310-a in conjunction with the perfusion angle calculator module 415.

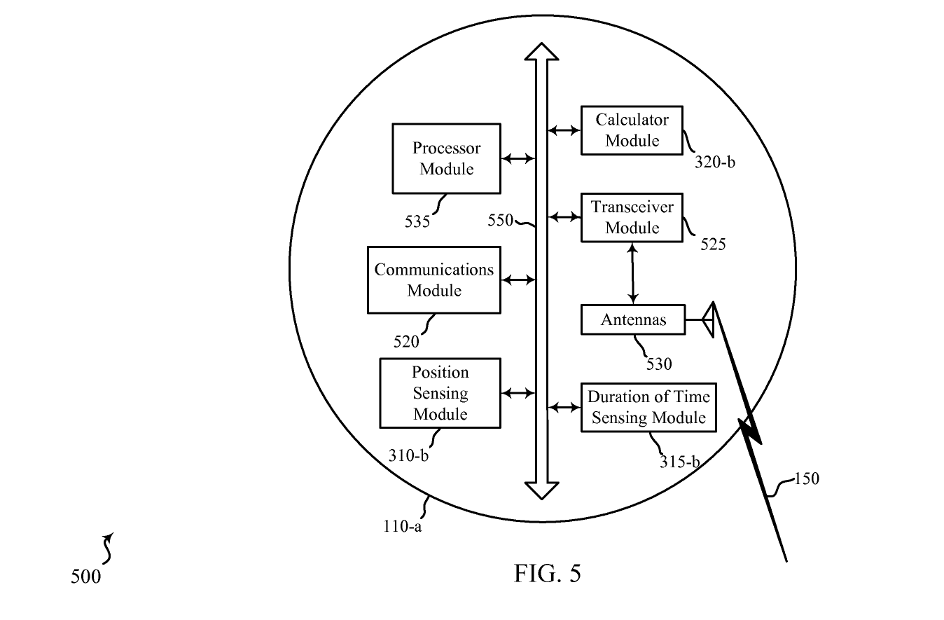

FIG. 5 shows a block diagram 500 of a sensor unit 110-a for use in remote physiological monitoring, in accordance with various aspects of the present disclosure. The sensor unit 110-a may have various configurations. The sensor unit 110-a may, in some examples, have an internal power supply (not shown), such as a small battery, to facilitate mobile operation. In some examples, the sensor unit 110-a may be an example of one or more aspects of one of the sensor units 110 and/or apparatus 305 described with reference to FIGS. 1 and/or 3. The sensor unit 110-a may be configured to implement at least some of the features and functions described with reference to FIGS. 1, 3 and/or 4.

The sensor unit 110-a may include a position sensing module 310-b, a processor module 535, a communications module 520, at least one transceiver module 525, at least one antenna (represented by antennas 530), a duration of time sensing module 315-b, or a calculator module 320-b. Each of these components may be in communication with each other, directly or indirectly, over one or more buses 550. The position sensing module 310-b, the duration of time sensing module 315-b, and the calculator module 320-b may be examples of the position sensing module 310, the duration of time sensing module 315, and the calculator module 320, respectively, of FIG. 3.

The processor module 535 may include an intelligent hardware device, e.g., a CPU, a microcontroller, an ASIC, etc. The processor module 535 may process information received through the transceiver module 525 or information to be sent to the transceiver module 525 for transmission through the antenna 530. The processor module 535 may handle, alone or in connection with the calculator module 320-b, various aspects of signal processing as well as determining patient position and duration of time spent in a position correlated with predetermined thresholds.

The transceiver module 525 may include a modem configured to modulate packets and provide the modulated packets to the antennas 530 for transmission, and to demodulate packets received from the antennas 530. The transceiver module 525 may, in some examples, be implemented as one or more transmitter modules and one or more separate receiver modules. The transceiver module 525 may support patient position-related communications. The transceiver module 525 may be configured to communicate bi-directionally, via the antennas 530 and communication link 145, with, for example, local computing devices 115, 120 and/or the remote computing device 140 (via network 125 and server 130 of FIG. 1). Communications through the transceiver module 525 may be coordinated, at least in part, by the communications module 520. While the sensor unit 110-a may include a single antenna 530, there may be examples in which the sensor unit 110-a may include multiple antennas 530.

The calculator module 320-b may be configured to perform or control some or all of the features or functions described with reference to FIGS. 1, 2, 3 and/or 4 related to determination of one or more patient position and durations of time spent in each of the one or more positions. For example, the calculator module 320-b may be configured to receive patient position data received from the position sensing module 310-b and duration of time data received from the duration of time sensing module 315-b. In some examples, the calculator module 320-b may determine whether a patient has occupied a given position beyond a predetermined turn time limit based, at least in part, on the monitored position data and duration of time data. The calculator module 320-b may determine that an alert should be issued based, at least in part, on the monitored position data and duration of time data, and the predetermined turn time and perfusion time thresholds. The calculated patient position and duration of time data (both the data, either processed or unprocessed, to which the position and duration of time data pertains as well as contextual data) may be transmitted to either a local computing device 115, 120 or a remote computing device 140. The calculator module 320-b, or portions of it, may include a processor, or some or all of the functions of the calculator module 320-b may be performed by the processor module 535 or in connection with the processor module 535. Additionally, the calculator module 320-b, or portions of it, may include a memory.

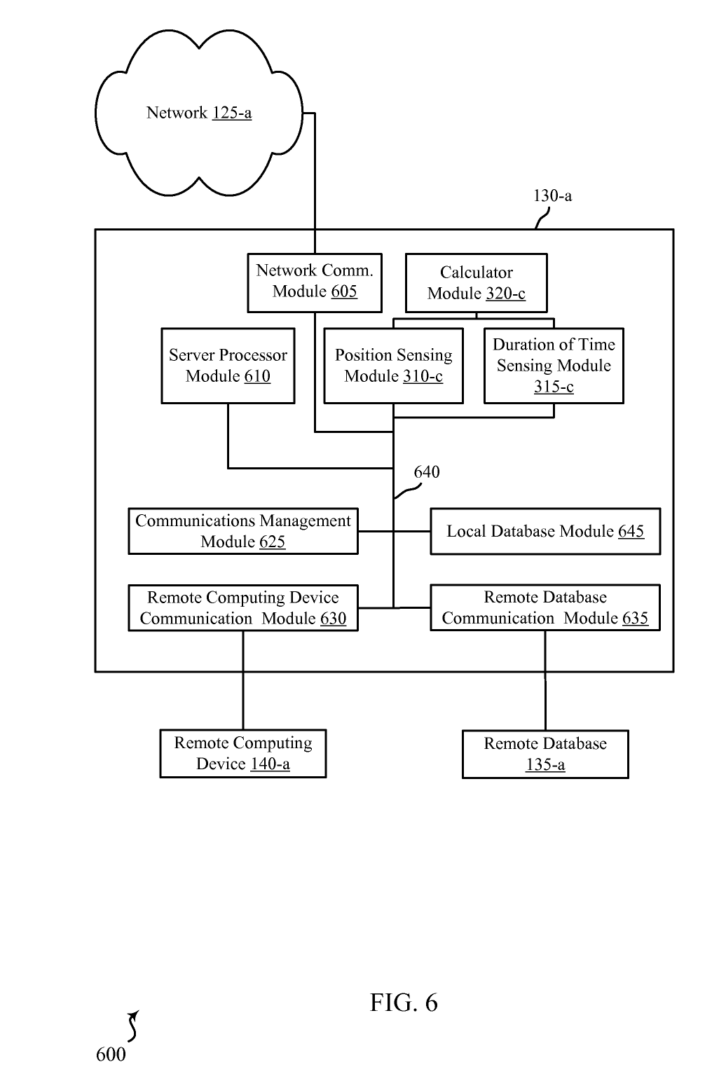

FIG. 6 shows a block diagram 600 of a server 130-a for use in determining position and duration of time spent in the position status of a patient, in accordance with various aspects of the present disclosure. In some examples, the server 130-a may be an example of aspects of the server 130 described with reference to FIG. 1. In other examples, the server 130-a may be implemented in either the local computing devices 115, 120 or the remote computing device 140 of FIG. 1. The server 130-a may be configured to implement or facilitate at least some of the features and functions described with reference to the server 130, the local computing devices 115, 120 and/or the remote computing device 140 of FIG. 1.

The server 130-a may include a server processor module 610, a local database module 645, and/or a communications management module 625. The server 130-a may also include one or more of a network communication module 605, a remote computing device communication module 630, and/or a remote database communication module 635. Each of these components may be in communication with each other, directly or indirectly, over one or more buses 640.

The server processor module 610 may include an intelligent hardware device, e.g., a central processing unit (CPU), a microcontroller, an ASIC, etc. The server processor module 610 may process information received through the one or more communication modules 605, 630, 635. The server processor module 610 may also process information to be sent to the one or more communication modules 605, 630, 635 for transmission. Communications received at or transmitted from the network communication module 605 may be received from or transmitted to sensor units 110, or local computing devices 115, 120 via network 125-a, which may be an example of the network 125 described in relation to FIG. 1. Communications received at or transmitted from the remote computing device communication module 630 may be received from or transmitted to remote computing device 140-a, which may be an example of the remote computing device 140 described in relation to FIG. 1. Communications received at or transmitted from the remote database communication module 635 may be received from or transmitted to remote database 135-a, which may be an example of the remote database 135 described in relation to FIG. 1. Additionally, a local database may be accessed and stored at the server 130-a. The local database module 645 is used to access and manage the local database, which may include data received from the sensor units 110, the local computing devices 115, 120, or the remote computing devices 140 (of FIG. 1).

The server 130-a may also include a calculator module 320-c, which may be an example of the calculator module 320 of apparatus 305 described in relation to FIGS. 3, 4 and/or 5. The calculator module 320-c may perform some or all of the features and functions described in relation to the calculator module 320, including selecting and obtaining from either the local database module 645 or the remote database 135-a data corresponding to the patient position and duration of time data, determining whether the patient has occupied a given position up to or beyond a predetermined turn time limit, and issuing an alert if the patient has met or exceeded the predetermined turn time limit based on patient position data and duration of time data collected by the sensor unit 110.

The server 130-a may further include a position sensing module 310-c and a duration of time sensing module 315-c, which may be examples of the position sensing module 310 and duration of time sensing module 315 described in relation to FIGS. 3, 4 and/or 5. The position sensing module 310-c and the duration of time sensing module 315-c may perform some or all of the features and functions described in relation to the position sensing module 310 and duration of time sensing module 315, including collecting data from one or more sensor units 110.

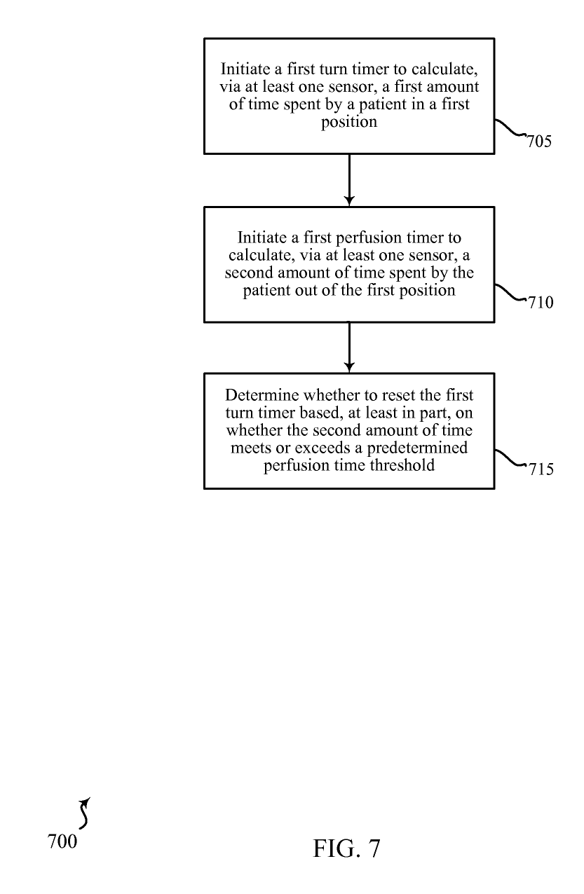

FIG. 7 is a flow chart illustrating an example of a method 700 of monitoring patient position and duration of time spent in a first position and out of the first position, in accordance with various aspects of the present disclosure. For clarity, the method 700 is described below with reference to aspects of one or more of the local computing devices 115, 120, remote computing device 140, and/or server 130 described with reference to FIGS. 1, and/or 6, or aspects of one or more of the apparatus 305 described with reference to FIGS. 3 and/or 4. In some examples, a local computing device, remote computing device or server such as one of the local computing devices 115, 120, remote computing device 140, server 130 and/or an apparatus such as one of the apparatuses 305 may execute one or more sets of codes to control the functional elements of the local computing device, remote computing device, server or apparatus to perform the functions described below.

At block 705, the method 700 may include initiating a first turn timer to calculate, via at least one sensor unit 110, a first amount of time spent by a patient in a first position. As discussed above, a patient may remain in any given position up to a predetermined turn time limit before the patient becomes at risk for developing pressure ulcers, or bedsores. Thus, a first turn timer is used to calculate, in conjunction with patient position data received from at least one sensor unit 110, the period of time spent by the patient in a first position.

At block 710, the method 700 may include initiating a first perfusion timer to calculate, via at least one sensor, a second amount of time spent by the patient out of the first position. As previously discussed, once a patient shifts out of a first position, a first perfusion timer may begin to operate to calculate, in conjunction with patient position data received from at least one sensor unit 110, the period of time spent by the patient out of the first position. The period of time spent out of the first position may be measured as a single, continuous period of time, or may be measured as a summation of a plurality of discrete periods of time spent out of the first position. In the latter example, a patient may have turned back to the first position one or more times. In addition, the period of time spent out of the first position may measure time spent in a second position, or may measure time spent in a plurality of positions.

At block 715, the method 700 may include determining whether to reset the first turn timer based at least in part on whether the second amount of time exceeds a predetermined perfusion time threshold. As discussed above, a patient must remain out of a first position for a predetermined period of time in order to allow the tissue previously under pressure in the first position to perfuse. Once the patient has remained out of the first position for an amount of time equal to or exceeding the predetermined perfusion time threshold, the first turn timer may be reset, such that the first turn timer may begin measuring the amount of time spent by the patient in a second position.

In some embodiments, the operations at blocks 705, 710 or 715 may be performed using the calculator module 320 described with reference to FIGS. 3, 4 and/or 5. Nevertheless, it should be noted that the method 700 is just one implementation and that the operations of the method 700 may be rearranged or otherwise modified such that other implementations are possible.

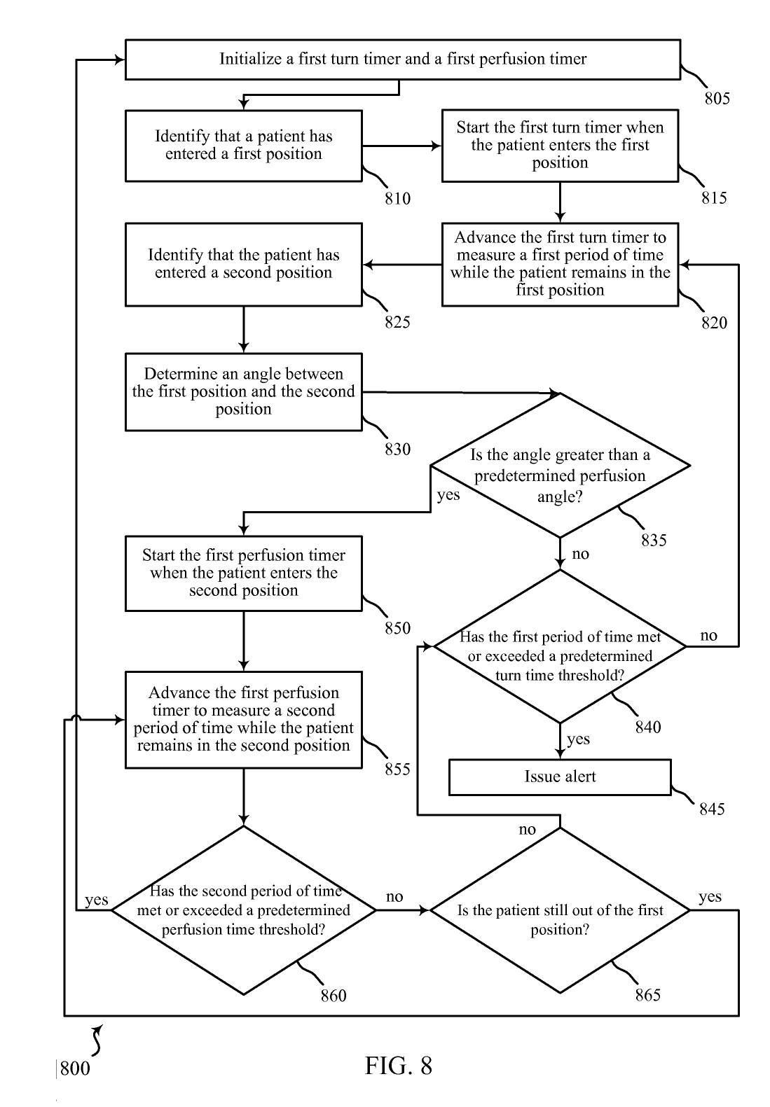

FIG. 8 is a flow chart illustrating an example of a method 800 for monitoring one or more patient positions and the duration of time spent in the one or more positions, in accordance with various aspects of the present disclosure. For clarity, the method 800 is described below with reference to aspects of one or more of the local computing devices 115, 120, remote computing device 140, and/or server 130 described with reference to FIGS. 1 and/or 6, or aspects of one or more of the apparatus 305 described with reference to FIGS. 3 and/or 4. In some examples, a local computing device, remote computing device or server such as one of the local computing devices 115, 120, remote computing device 140, server 130 and/or an apparatus such as one of the apparatuses 305 may execute one or more sets of codes to control the functional elements of the local computing device, remote computing device, server or apparatus to perform the functions described below. In other embodiments, the method 800 may be carried out entirely at the one or more sensor units 110.

The method 800 may be used, for example, to monitor a patient's one or more positions, via one or more sensors, and to concurrently monitor the amount of time spent by the patient in each position, in order to provide alerts to the patient or caregiver when the patient is at risk of developing pressure ulcers. In some embodiments, the patient may be monitored in a hospital, a hospice or other healthcare related facility. In other embodiments, the patient may be monitored at home and the patient's position data and duration of time data may be streamed to the location of the healthcare provider. Based on the received data, an alert may be issued indicating to the patient and/or caregiver that the patient should be turned to a different position to allow for tissue perfusion and to avoid the development of bedsores.

As shown in FIG. 8, at step 805, the method 800 includes initializing a first turn timer and a first perfusion timer. In some embodiments, a single turn timer and perfusion timer may operate to measure all patient positions. In alternative embodiments, a separate turn timer and perfusion timer may be operated for each of a plurality of discrete zones on a patient's body. For example, a first turn timer and perfusion timer may monitor patient position for a patient's shoulder blades, while a second turn timer and perfusion timer may monitor patient position for a patient's tailbone. Thus, if the patient shifts in position such that tissue at one portion of his body, such as his shoulder, is allowed to perfuse, but tissue at another portion of his body, such as his tailbone, is still under pressure, the series of turn timers and perfusion timers may monitor risk of bedsore development for each zone of patient tissue.

At step 810, the method 800 includes identifying that a patient has entered a first position. This identification may be achieved, as described above with reference to FIGS. 3 and 4, by collecting patient position data at one or more sensor units 110, and communicating that patient position data to position sensing module 310. The first position may be any seated or reclined position, such that patient risk of bedsore development may be monitored regardless of whether a patient is lying in a hospital bed, or is sitting in a chair at home, or is in any other location. At step 815, the method 800 includes starting the first turn timer when the patient enters the first position, and at step 820, advancing the first turn timer to measure a first period of time, the first period of time corresponding to the duration of time spent by the patient in the first position. In this way, the one or more sensor units 110, in conjunction with the position sensing module 310, may measure a period of time spent by the patient in a first position. As discussed with regard to FIGS. 2A-D, the turn timer may advance at a positive rate, which rate may vary based on individual patient parameters, for the duration of time spent by the patient in the first position.

At step 825, the method 800 includes identifying that the patient has entered a second position. Again, this identification may be achieved by receiving patient position data from position sensing module 310 via one or more sensor units 110.

At step 830, the method 800 may include determining an angle between the first position and the second position. As described above with regard to FIG. 4, a perfusion angle calculator module 415 may collect patient position data from position sensing module 310-a to determine whether the patient has in fact shifted from the first position to a second position, on the basis of whether the angle between the first position and the second position is greater than a predetermined perfusion angle. The perfusion angle may be patient-specific, and may be determined based on the individual patient's physiological needs to ensure that tissue put under pressure in a first position is sufficiently free of pressure in a second position such that the tissue may properly perfuse. For example, a patient with a smaller body mass index may require a smaller predetermined perfusion angle in order to relieve the pressure on tissue previously under pressure, while a patient with a larger body mass index may be required to turn at a greater angle to ensure that all tissue previously under pressure is now able to perfuse. As discussed above, in order to avoid the development of bedsores, tissue must be allowed sufficient time to perfuse such that blood may return to the tissue. Thus, if at step 835 it is determined that the angle between the first position and the second position is greater than a predetermined perfusion angle, the perfusion timer will start when the patient enters the second position, at step 850. In this way, the perfusion timer may begin to measure the period of time spent by the patient in the second position.

The perfusion timer will continue to advance at step 855 while the patient remains in the second position. At step 860, the method 800 may include determining whether the perfusion timer has exceeded a predetermined perfusion time threshold. As previously discussed, a patient must remain out of a first position for a sufficient period of time to allow the patient's tissue previously placed under pressure in the first position to perfuse. Thus, if the patient has occupied the second position for a sufficient period of time with regard to the predetermined perfusion time threshold such that the tissue has properly perfused, the method 800 will return to step 805, in which the first turn timer and the first perfusion timer will each reset, or initialize. As previously discussed with regard to FIGS. 2A-D, in some embodiments the perfusion timer may measure one continuous period of time spent by the patient out of a first position in order to determine whether a perfusion time limit has been met or surpassed. In other embodiments, the perfusion timer may measure a summation of a plurality of time periods spent by the patient out of the first position. For example, the patient may spend 40 minutes in a first position, may turn to a second position for 10 minutes, may return to the first position for 20 minutes, may roll back to the second position for 5 minutes, may shift back to the first position for 15 minutes, and may roll back to the second position for an additional 5 minutes such that, in the aggregate, the patient has spent 20 minutes in the second position such that a predetermined perfusion time threshold of 20 minutes has been met.

While described with reference to a first position and a second position in this embodiment, in alternate embodiments perfusion time may be measured with regard to an amount of time spent by the patient in any position that is out of the first position. For example, the patient may shift from a first position to a second position, and then may shift from a second position to a third position, and so on, such that the perfusion timer measures the totality of time spent by the patient in any position that is out of the first position.

If, at step 860, it is determined that the perfusion timer has not met or exceeded a predetermined perfusion time threshold, it may then be determined at step 865 whether the patient is still out of the first position. If the answer is in the affirmative, the perfusion timer will continue to advance while the patient remains in the second position, as shown in step 855. If, in the alternative, it is determined at step 865 that the patient is not still out of the first position, but has instead returned to the first position, it will be determined at step 840 whether the first period of time has met or exceeded a predetermined turn time threshold. As previously discussed, the turn timer may continue to operate at a positive rate for so long as the patient is in a first position. The turn timer may then pause, or in some embodiments may operate at a negative rate, while the patient is out of the first position, and may continue to operate at a positive rate when the patient returns to the first position. Thus, if it is determined at step 865 that the patient has returned to the first position, it may then be determined at step 840 whether the turn timer has now met or exceeded the predetermined turn time threshold. If it is determined that the patient has occupied the first position, either continuously, or in the aggregate, for a period of time meeting or exceeding the predetermined turn time threshold, an alert may be issued at step 845. The alert may be issued from the one or more sensor units 110, or may be transmitted to a local or remote computing device 115, 120, 140 in order to notify the patient or a caregiver that the patient should be shifted to a new position in order to avoid the development of bedsores. In the alternative, if the patient has not spent enough time in the first position to exceed the predetermined turn time limit, the method 800 will return to step 820, wherein the first turn timer will continue to operate to measure a first period of time spent in the first position.

Returning to step 835, if it is determined that the angle between the first position and the second position is not greater than a predetermined perfusion angle, in other words, that the patient has not shifted out of the first position sufficiently to allow the tissue placed under pressure in the first position to perfuse, it may be determined that the patient is still in the first position for purposes of tissue pressure. In this case, the first turn timer will have continued to operate at a positive rate for the entire time, despite the patient having shifted slightly within the first position. It may then be determined at step 840 whether the first period of time has met or exceeded a predetermined turn time threshold. Again, the patient may only occupy a first position, either continuously or in the aggregate, for a predetermined turn time limit before the patient becomes at risk for developing bedsores due to lack of blood flow to the tissue. Thus, if it is determined at step 840 that the patient has occupied the first position for a period of time meeting or exceeding the predetermined turn time threshold, an alert is issued at step 845 to notify the patient and/or caregiver that the patient should be turned to a new position. If, in the alternative, the patient has not occupied the first position for a period of time exceeding the predetermined turn time limit as determined at step 840, the method 800 will return to step 820, in which the turn timer will continue to advance to measure the period of time spent by the patient in the first position.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

The detailed description set forth above in connection with the appended drawings describes exemplary embodiments and does not represent the only embodiments that may be implemented or that are within the scope of the claims. The term “exemplary” used throughout this description means “serving as an example, instance, or illustration,” and not “preferred” or “advantageous over other embodiments.” The detailed description includes specific details for the purpose of providing an understanding of the described techniques. These techniques, however, may be practiced without these specific details. In some instances, well-known structures and devices are shown in block diagram form in order to avoid obscuring the concepts of the described embodiments.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitate transfer of a computer program from one place to another. A storage medium may be any medium that may be accessed by a general purpose or special purpose computer. By way of example, and not limitation, a computer-readable medium may comprise RAM, ROM, EEPROM, CD-ROM or other optical disk storage, magnetic disk storage or other magnetic storage devices, or any other medium that may be used to carry or store desired computer-readable program code in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

1. A method of preventing pressure ulcers, comprising:
initiating a first turn timer to calculate, via at least one sensor, a first amount of time spent by a patient in a first position;
initiating a first perfusion timer to calculate, via at least one sensor, a second amount of time spent by the patient out of the first position; and
determining whether to reset the first turn timer based, at least in part, on whether the second amount of time meets or exceeds a predetermined perfusion time threshold.

2. The method of claim 1, further comprising:
issuing an alert based on the first amount of time meeting or exceeding a first predetermined turn time threshold.

3. The method of claim 1, further comprising:
resetting the first perfusion timer based on the patient returning to the first position.

4. The method of claim 1, further comprising:
advancing the first turn timer linearly, non-linearly, or via a step function; and
advancing the first perfusion timer linearly, non-linearly, or via a step function.

5. The method of claim 1, further comprising:
advancing the first turn timer at a turn timer rate; and
advancing the first perfusion timer at a perfusion timer rate.

6. The method of claim 5, wherein advancing the first turn timer comprises:
setting the turn timer rate to have a positive slope when the patient is in the first position; and
setting the turn timer rate to be substantially zero when the patient is out of the first position.

7. The method of claim 5, wherein advancing the first turn timer comprises:
setting the turn timer rate to have a positive slope when the patient is in the first position; and
setting the turn timer rate to have a negative slope when the patient is out of the first position.

8. The method of claim 5, wherein advancing the first perfusion timer comprises:
setting the perfusion timer rate to have a positive slope when the patient is out of the first position; and
setting the perfusion timer rate to be substantially equal to zero when the patient is in the first position.

9. The method of claim 5, wherein the turn timer rate and the perfusion timer rate are different.

10. The method of claim 1, further comprising:
initiating a second turn timer to calculate, via at least one sensor, a third amount of time spent by a patient in a second position;
initiating a second perfusion timer to calculate, via at least one sensor, a second amount of time spent by the patient out of the second position; and
determining whether to reset the second turn timer based, at least in part, on whether the second amount of time meets or exceeds a predetermined perfusion time threshold.

11. The method of claim 10, wherein:
the initiating the first perfusion timer is based on the patient moving out of the first position; and
the initiating the second perfusion timer is based on the patient moving out of the second position.

12. The method of claim 1, further comprising:
determining that a patient has moved out of the first position by identifying that an angle between the first position and a current position of the patient is greater than a predetermined perfusion angle.

13. The method of claim 12 further comprising:
modifying the predetermined perfusion angle based on patient physiological data.

14. The method of claim 1, further comprising:
using at least two sensors to determine a position of the patient, each of the at least two sensors positioned at a discrete zone on the patient's body.

15. A physiological monitoring device, comprising:
a processor configured to
receive, via at least one sensor, indicators of a position of a patient;
determine, using the indicators, whether the patient is in a first position or has moved out of the first position, a first amount of time being spent by the patient in the first position, and a second amount of time being spent by the patient out of the first position; and
determining whether to reset the first amount of time based, at least in part, on the second amount of time meeting or exceeding a first predetermined perfusion time threshold.

16. The device of claim 15, wherein the processor is further configured to:
operate a turn timer to determine the first amount of time being spent by the patient in the first position; and
operate a perfusion timer to determine the second amount of time being spent by the patient out of the first position.

17. The device of claim 15, wherein the processor is further configured to:
determine an angle between the first position and a current position of the patient; and
determine the second amount of time being spent by the patient out of the first position while the angle is greater than a predetermined perfusion angle.

18. The device of claim 15, further comprising:
an alert output configured to output an alert based on the first amount of time exceeding a predetermined turn time threshold,
wherein the alert output is configured to output any one or more of an audible, visual or haptic alert.

19. The device of claim 15, wherein the processor is further configured to:
receive indications from at least two sensors, each of the at least two sensors positioned at a discrete zone on the patient's body.

20. A non-transitory computer-readable medium storing computer-executable code, the code executable by a processor to:
initiate a first turn timer to calculate, via at least one sensor, a first amount of time spent by a patient in a first position;
initiate a first perfusion timer to calculate, via at least one sensor, a second amount of time spent by the patient out of the first position; and
determine whether to reset the first turn timer based, at least in part, on whether the second amount of time meets or exceeds a predetermined perfusion time threshold.
